(19) United States
(12) Patent Application Publication
(10) Pub. No.: US 2006/0275331 A1
(43) Pub. Date: Dec. 7, 2006

(54) Pharmaceutical composition, method of manufacturing and therapeutic use thereof

(76) Inventors: Borek Zaludek, Brno (CZ); Jan Boleslav, Tisnov (CZ); Vladimir Kysilka, Brno (CZ); Jirl Verner, Brno (CZ); Hana Brzobohata, Brno (CZ); Bozena Matejkova, Brno (CZ); Milan Kakrda, Brno (CZ)

Correspondence Address: Peter C Michalos;Notaro & Michalos, Suite 110, 100 Dutch Hill Road, Orangeburg, NY, 10962-2100, US

(21) Appl. No.: 10/569,866

(22) PCT Filed: Aug. 31, 2004
(86) PCT No.: PCT/CZ04/00053

§ 371 Date: Feb. 28, 2006

(51) Int. Cl. A61K 31/282; A61K 31/05; A61K 9/00

(52) U.S. Cl. 424/400; 514/492; 514/732

(57) ABSTRACT

The invention relates to a pharmaceutical composition prepared by freeze-drying in vacuo, containing oxaliplatin as the active component and a pharmaceutically acceptable carrier, wherein the carrier is at least one alcoholic sugar of non-animal origin, the weight ratio of oxaliplatin to the alcoholic sugar of non-animal origin or alcoholic sugars of non-animal origin being 1:3 to 1:7. The invention also relates to the method of manufacturing of said composition and to the use of this composition in the treatment of tumors sensitive to oxaliplatin.

### FIELD OF THE INVENTION

The invention relates to a pharmaceutical composition which is prepared by freeze-drying and contains oxaliplatin as the active component and a pharmaceutically acceptable carrier, application of this composition reducing the risk of viral contamination, especially by causers of animal spongiform encephalopathy.

### BACKGROUND OF THE INVENTION

Oxaliplatin (named according to the INN nomenclature as oxaliplatinum) has been prepared for the first time in the optically pure form in 1978 by isolation from a mixture of isomers [J. Clin. Hematol. Oncol. 1977, 7(1), 197-210]. Oxaliplatin is chemically the {trans-(−)-1,2-cyclohexanediamine}-(oxalato)platinum(II) complex of formula I

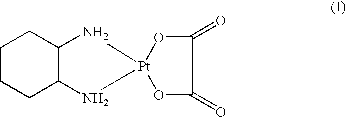

Oxaliplatin is a cytostatic agent used in treatment of testicular and ovarial tumors, malignant melanoma, bronchogenic carcinoma and especially of metastazing colon carcinoma in combination with fluoropyrimidines. In comparison with the hitherto used cisplatinum, oxaliplatin exhibits lower nefrotoxicity and a broader spectrum of antitumor activity. Oxaliplatin is generally applied in doses 100 to 135 mg/m² in the form of 2 to 6 hours′ infusion. The infusion solution is prepared by dilution of the final drug form with 5% aqueous solution of glucose.

Within the framework of the prior art there was described a series of liquid drug forms of oxaliplatin based on its aqueous solutions which however for their insufficient stability so far have not found application. The cause of this instability is the known fact that oxaliplatin hydrolytically decomposes under formation of toxic platinum aquacomplexes. The only stable drug form of oxaliplatin known so far is a powder composition obtained by freeze-drying in vacuo (lyophilization). Beside oxaliplatin, this dry powdery composition contains a carrier consisting of lactose which ensures the cohesion of the dry oxaliplatin matrix formed by freeze-drying in vacuo and prevents its cracking, which otherwise would result in escape of part of the oxaliplatin from vials in which the freeze-drying takes place. The substantial drawback of the hitherto used lactose carrier, which is an animal product, is the risk of viral contamination of the pharmaceutical composition by viruses that may be present in lactose. Recently, reduction of the risk of such viral contamination, especially of transfer of animal spongiform encephalopathy, represents a current problem concerning most drug forms that contain auxiliary components of animal, particularly bovine origin. Lactose, hitherto employed in oxaliplatin pharmaceutical compositions, exhibits very good cryoprotective effects and its replacement with another suitable carrier under preservation of properties of oxaliplatin lyophilizate and economy of the freeze-drying process so far has not been satisfactorily solved.

### SUMMARY OF THE INVENTION

The above-mentioned desired replacement of the lactose carrier with another carrier which in principle eliminates the mentioned risk of viral contamination is solved by the pharmaceutical composition according to the present invention, prepared by freeze-drying in vacuo, that contains oxaliplatin as the active component together with a pharmaceutically acceptable carrier, the said composition being characterized in that it contains at least one alcoholic sugar of non-animal origin as a carrier, the ratio of oxaliplatin to this alcoholic sugar or alcoholic sugars of non-animal origin being 1:3 to 1:7 by weight.

Preferably, the pharmaceutical composition according to the invention contains oxaliplatin and an alcoholic sugar of non-animal origin or alcoholic sugars of non-animal origin in a ratio 1:5 by weight.

In a pharmaceutical composition according to the invention, the preferred alcoholic sugar of non-animal origin is mannitol.

The present invention also relates to the method of manufacturing of the above mentioned pharmaceutical composition, characterized in that a sterile aqueous solution of oxaliplatin and of at least one alcoholic sugar of non-animal origin, containing oxaliplatin and an alcoholic sugar of non-animal origin or alcoholic sugars of no-animal origin in a weight ratio 1:3 to 1:7 with total concentration of the mentioned compounds 2.8 to 3.2% by weight, is introduced into a vial in a volume equal to at most 60% of the available vial volume, whereupon the content of the vial is cooled to 2 to 8° C., then freezed under linear temperature drop of 0.1 to 0.5° C./min to a final temperature of −35 to −45° C., left aside at this temperature for 1 to 6 hours and then subjected to freeze-drying in vacuo.

Finally, the invention also relates to the above mentioned pharmaceutical composition for application in treatment of tumors sensitive to oxaliplatin.

After dissolution in a suitable pharmaceutically acceptable solvent, the pharmaceutical composition according to this invention affords a clear solution which contains neither undissolved material nor turbidity and which is thus particularly suitable for parenteral application. The composition according to the invention can be readily prepared, is highly stable and its application does not represent any risk of viral contamination.

The method according to the present invention solves problems that would occur in the case of mere replacement of lactose by mannitol without changing the existing mode of working with lactose. During workup of solutions in which lactose is simply replaced with mannitol, the lyophilizate often escapes from the vials and the glass vials crack as the result of increased mechanical tension due to the changing volume of the freezed solution, which is particularly manifested by falling away of the vial bottom during the freeze-drying procedure. It has been found that the temperature mode of the freezing procedure according to the invention significantly eliminates the mentioned vial cracking in the course of the freeze-drying procedure. Thus, it was found that in order to achieve an optimal freeze-drying rate the total concentration of oxaliplatin and of at least one alcoholic sugar in the aqueous solution before the freeze-drying must be 2.8 to 3.2% by weight. Further, it was found that the vials can be filled with the sterile solution of oxaliplatin and mannitol in the mentioned weight ratio in a volume up to 60% of the available vial volume without change of the lyophilizate quality, without escape of the lyophilizate from the vials, and without cracking of the vials, which represents a very advantageous solution from the viewpoint of utilization of capacity of the freeze-drying equipment.

For its properties, the pharmaceutical composition according to the invention is particularly suitable for application in treatment of tumors sensitive to oxaliplatin.

In the following part of the description the invention will be explained in detail using individual examples of execution, that are only illustrative, without limiting in any way the scope of the invention.

### EXAMPLES

### Example 1

Under GMP conditions, 0.50 kg of oxaliplatin (pharmacopoeia quality) and 2.50 kg of mannitol (pharmacopoeia quality) are gradually dissolved at 20 to 25° C. in 70 kg of water. The obtained solution is sterilized by filtration through a 0.22 μm filter and filled into colourless clear vials for antibiotics of the first hydrolytic class, type 20 H (Saint Gobain Desjonqueres) the amount of the solution corresponding to 50 mg of the dissolved active component per vial. The filled vials are equipped with stoppers for freeze-drying and placed into a freeze-drying chamber, pre-cooled to 5° C. After thermal equilibration of the solution in the vial with the environment, the solution in the vial is slowly freezed (linear temperature drop of 0.2° C./min) to the final temperature of −40° C. The frozen solution is left at this temperature for 4 h, whereupon it is subjected to freeze-drying in vacuo.

The obtained lyophilizate has a white compact form and contains 0.8% by weight of water. Dissolution of the obtained material gives a clear solution, in accord with Ph. Eur. Art. 2.2.1.

### Example 2

Under GMP conditions, 0.50 kg of oxaliplatin (pharmacopoeia quality) and 2.50 kg of mannitol (pharmacopoeia quality) are gradually dissolved at 20 to 25° C. in 70 kg of water. The obtained solution is sterilized by filtration through a 0.22 μm filter and filled into colourless clear vials for antibiotics of the first hydrolytic class, type 20 H (Saint Gobain Desjonqueres) the amount of the solution corresponding to 100 mg of the dissolved active component per vial. The filled vials are equipped with stoppers for freeze-drying and placed into a freeze-drying chamber, pre-cooled to 5° C. After thermal equilibration of the solution in the vial with the environment, the solution in the vial is slowly freezed (linear temperature drop of 0.2° C./min) to the final temperature of −40° C. The frozen solution is left at this temperature for 4 h, whereupon it is subjected to freeze-drying in vacuo.

The obtained lyophilizate has a white compact form and contains 1.0% by weight of water. Dissolution of the obtained material gives a clear solution, in accord with Ph. Eur. Art. 2.2.1.

### Example 3

This Example studies the stability of the pharmaceutical composition prepared in Example 1 when stored at 40° C. and 75% relative humidity. The obtained results are given in Table 1 below.

TABLE 1

| Parameter evaluated | At the beginning of storage | After 6 months' storage |
|---|---|---|
| Appearance of lyophilizate | White, compact | White, compact |
| Chromatographic purity of | | |
| lyophilizate: | | |
| Oxalic acid (%) | 0.10 | 0.08 |
| Total impurities (%) | 0.11 | 0.16 |
| L-isomer (%) | <0.1 | <0.1 |
| Water content in lyophilizate (%) | 0.8 | 1.0 |
| Appearance of solution after | Clear according to Ph. Eur. | Clear according to Ph. |
| dissolution of lyophilizate (3% | Art. 2.2.1 | Eur. Art. 2.2.1 |
| aqueous solution) | | |
| Contamination of lyophilizate | Without visible particles | Without visible particles |
| solution | | |

The percentages given in Table 1 are % by weight.

### Example 4

This Example studies the stability of the pharmaceutical composition prepared in Example 2 when stored at 40° C. and 75% relative humidity. The obtained results are given in Table 2 below.

TABLE 2

| Parameter evaluated | At the beginning of storage | After 6 months' storage |
|---|---|---|
| Appearance of lyophilizate | White, compact | White, compact |
| Chromatographic purity of | | |
| lyophilizate: | | |
| Oxalic acid (%) | 0.10 | <0.1 |
| Total impurities (%) | 0.11 | 0.12 |
| L-isomer (%) | <0.1 | <0.1 |
| Water content in lyophilizate (%) | 1.0 | 1.1 |
| Appearance of solution after | Clear according to Ph. Eur. | Clear according to Ph. Eur. |
| dissolution of lyophilizate (3% | Art. 2.2.1 | Art. 2.2.1 |
| aqueous solution) | | |
| Contamination of lyophilizate | Without visible particles | Without visible particles |
| solution | | |

The percentages given in Table 2 are % by weight.

1. A pharmaceutical composition prepared by freeze-drying in vacuo, containing oxaliplatin as the active component and a pharmaceutically acceptable carrier, characterized in that the carrier is at least one alcoholic sugar of non-animal origin, the weight ratio of oxaliplatin to the alcoholic sugar of non-animal origin or alcoholic sugars of non-animal origin being 1:3 to 1:7.

2. The pharmaceutical composition according to claim 1, characterized in that it contains oxaliplatin and an alcoholic sugar of non-animal origin or alcoholic sugars of non-animal origin in a weight ratio 1:5.

3. The pharmaceutical composition according to claim 1, characterized in that the alcoholic sugar of non-animal origin is mannitol.

4. A method of manufacturing of pharmaceutical composition according to claim 1, characterized in that a sterile aqueous solution of oxaliplatin and of at least one alcoholic sugar of non-animal origin, containing oxaliplatin and an alcoholic sugar of non-animal origin or alcoholic sugars of non-animal origin in a weight ratio 1:3 to 1:7, with total concentration of the mentioned compounds 2.8 to 3.2% by weight, is introduced into a vial in a volume amount equal to at most 60 vol % of the available vial volume, whereupon the content of the vial is cooled to a temperature of 2 to 8° C., then freezed under linear temperature drop of 0.1 to 0.5° C./min to a final temperature of −35 to −45° C., left at this temperature for 1 to 6 hours and then subjected to freeze-drying in vacuo.

5. The pharmaceutical composition according to claim 1 for use in the treatment of tumors sensitive to oxaliplatin.
